(19) United States
(12) Patent Application Publication
(10) Pub. No.: US 2026/0130702 A1
(43) Pub. Date: May 14, 2026

(54) POWER SUPPLY DEVICE AND ABLATION SYSTEM

(71) Applicant: Japan Lifeline Co., Ltd., Tokyo (JP)

(72) Inventors: Takuya HIRAO, Tokyo (JP); Shuichi HORIUCHI, Tokyo (JP)

(21) Appl. No.: 19/481,860

(22) PCT Filed: May 20, 2024
(86) PCT No.: PCT/JP2024/018513

§ 371 Date: Nov. 5, 2025

(30) Foreign Application Priority Data
Sep. 22, 2023 (JP) 2023-158608

(51) Int. Cl. A61B 18/00

(52) U.S. Cl. CPC A61B 18/00; A61B 2018/0016; A61B 2018/00267; A61B 2018/00577; A61B 2018/00613; A61B 2018/00702; A61B 2018/00761; A61B 2018/00767

(57) ABSTRACT

A power supply device includes: a power source unit electrically connected to a catheter including a plurality of electrodes, and configured to apply voltage to the plurality of electrodes; and a control unit configured to control the power source unit to continuously apply voltage a plurality of times to one or more electrodes of the plurality of electrodes, and then continuously apply voltage a plurality of times to another one or more electrodes of the plurality of electrodes.

### CROSS-REFERENCE TO RELATED APPLICATIONS

This application is based upon and claims the benefit of priority from the prior Japanese Patent Application No. 2023-158608, filed on Sep. 22, 2023, and the International Patent Application No. PCT/JP 2024/018513, filed on May 20, 2024, the entire content of each of which is incorporated herein by reference.

### BACKGROUND

### Field of the Invention

The present disclosure relates to a power supply device and an ablation system.

### Description of the Related Art

Patent Document 1 discloses an ablation system including an ablation catheter and a pulse waveform generator that delivers voltage pulses to the ablation catheter.

Patent Document 1: JP2019-500170 A

As a result of intensive studies, the present inventors have conceived a novel technique related to catheter ablation procedures.

### SUMMARY OF THE INVENTION

In view of the above-described circumstances, an object of the present disclosure is to provide a novel technique related to catheter ablation procedures.

An aspect of the present disclosure relates to a power supply device. The power supply device includes: a power source unit electrically connected to a catheter including a plurality of electrodes, and configured to apply voltage to the plurality of electrodes; and a control unit configured to control the power source unit to continuously apply voltage a plurality of times to one or more electrodes of the plurality of electrodes, and then continuously apply voltage a plurality of times to another one or more electrodes of the plurality of electrodes.

Another aspect of the present disclosure relates to an ablation system. The ablation system includes: a catheter including a plurality of electrodes; and the above-described power supply device.

Any arbitrary combination of the above components, as well as conversions of the expressions of the present disclosure between methods, devices, systems, and the like, are also valid aspects of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a schematic view of an ablation system according to an embodiment;

FIG. 2 is a perspective view of an electrode assembly;

FIG. 3 is a diagram illustrating a first example of a timing of voltage application to each electrode; and

FIG. 4 is a diagram illustrating a second example of a timing of voltage application to each electrode.

### DETAILED DESCRIPTION OF THE INVENTION

Hereinafter, the present disclosure will be described with reference to the drawings based on preferred embodiments. The embodiments are not intended to limit the present disclosure but are illustrative, and all features and combinations thereof described in the embodiments are not necessarily essential to the present disclosure. The same or equivalent components, members, and processes shown in the respective drawings are denoted by the same reference numerals, and repeated explanations are omitted as appropriate.

In addition, the scale and shape of each part shown in the drawings are set for convenience to facilitate understanding and are not to be construed as limiting unless specifically stated otherwise. Furthermore, when terms such as “first” and “second” are used in this specification or the claims, such terms do not indicate any order or importance unless otherwise specified, but are used merely to distinguish one component from another. Some members that are not important for explaining the embodiments are omitted from the drawings.

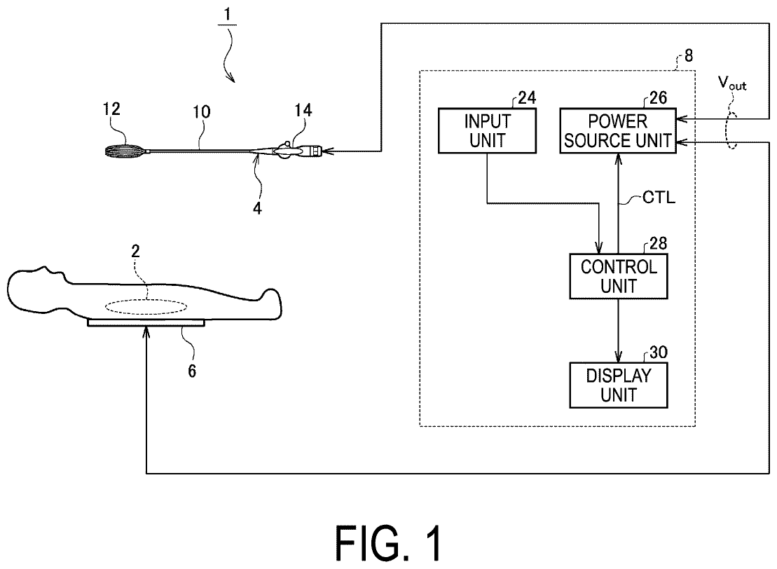

FIG. 1 is a schematic view of an ablation system 1 according to an embodiment. FIG. 1 illustrates some components of the ablation system 1 as functional blocks. At least some of these functional blocks can be implemented as a hardware configuration using an element and a circuit including a CPU and a memory of a computer, and as a software configuration using a computer program and the like. It will be understood by those skilled in the art that these functional blocks can be implemented in various forms by a combination of hardware and software.

The ablation system 1 performs a predetermined ablation on an affected area 2 of the patient. Examples of the affected area 2 include organs in which arrhythmia occurs. Note that the ablation system 1 may be used for ablation on other affected areas 2. The ablation system 1 includes a catheter 4, a counter electrode plate 6, and a power supply device 8.

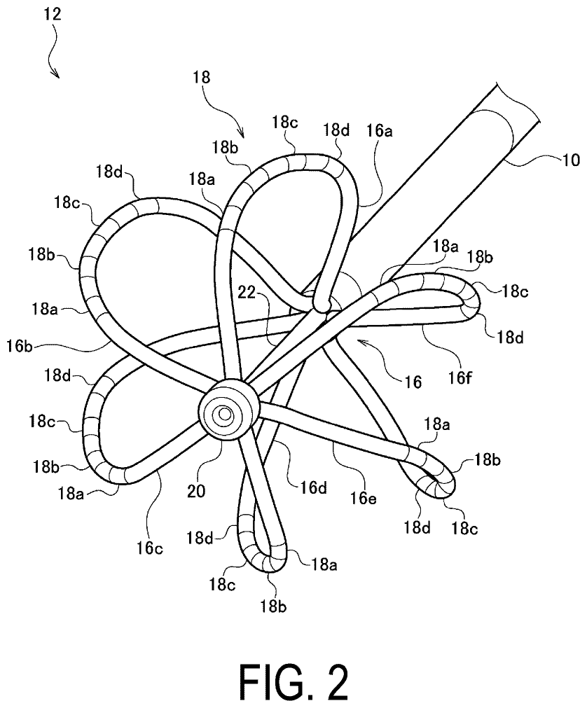

The catheter 4, as an example, includes a shaft 10, an electrode assembly 12, and a handle 14. The shaft 10 is composed of a flexible tubular member, and at least the distal end side is inserted into the body of the patient. The shaft 10 is composed of a publicly known flexible material including resins such as polyolefin, polytetrafluoroethylene, polyether block amide, and polyamide. The shaft 10 has a multiple-lumen structure including a plurality of lumens, for example. Various thin wires (not illustrated) such as conductive wires and operating wires, an inner tube 22 described later (see FIG. 2), and the like are inserted into a lumen.

The electrode assembly 12 is provided at the distal end of the shaft 10. FIG. 2 is a perspective view of the electrode assembly 12. The electrode assembly 12 includes a plurality of splines 16 and a plurality of electrodes 18. Note that FIG. 1 illustrates a state where the spline 16 is folded, and FIG. 2 illustrates a state where the spline 16 is unfolded.

Each spline 16 is a linear member extending in the axis direction of the shaft 10, and is composed of the same flexible material as that of the shaft 10. As an example, the electrode assembly 12 illustrated in FIG. 2 includes a first spline 16a, a second spline 16b, a third spline 16c, a fourth spline 16d, a fifth spline 16e and a sixth spline 16f, but the number of the splines 16 is not limited to six as long as a plurality of splines 16 is provided. In the present disclosure, the first spline 16a to the sixth spline 16f may be simply referred to as “spline 16” when it is unnecessary to distinguish among them.

Each spline 16 is disposed in the axial direction of the shaft 10 with intervals therebetween. The distal end of each spline 16 is connected to a distal end tip 20. The proximal end of each spline 16 is inserted into the shaft 10 from the distal end of the shaft 10 and fixed to the shaft 10. The distal end of the inner tube 22 is connected to the distal end tip 20. The inner tube 22 is passed through the lumen of the shaft 10, and the proximal end is connected to the handle 14. The inner tube 22 can be moved to the distal end side and the proximal end side of the shaft 10 by operating the handle 14.

When the inner tube 22 is drawn to the proximal end side of the shaft 10 with each spline 16 linearly extended, the distal end tip 20 is displaced to the proximal end side of the shaft 10. As a result, each spline 16 curves outward in a bulging manner, and the electrode assembly 12 is set to a basket shape. When the inner tube 22 is pushed to the distal end side of the shaft 10 while each spline 16 is in a curved state, the distal end tip 20 is displaced to the distal end side of the shaft 10. As a result, each spline 16 is set to a linear shape, and the electrode assembly 12 is folded. Note that the term “basket shape” is derived from the resemblance of the shapes of the plurality of splines 16 to the curved patterns on the surface of a basketball.

Each spline 16 is provided with the plurality of electrodes 18. The plurality of electrodes 18 is disposed at predetermined intervals therebetween in the longitudinal direction of the spline 16. Each electrode 18 has a ring shape, and is made of a metal having good electrical conductivity, such as platinum, gold, silver, copper, aluminum, or stainless steel, or an alloy thereof. As an example, the electrode assembly 12 illustrated in FIG. 2 includes a first electrode 18a, a second electrode 18b, a third electrode 18c, and a fourth electrode 18d on each spline 16, but the number of electrodes 18 is not limited to four as long as at least one electrode 18 is provided. In the present disclosure, the first electrode 18a to the fourth electrode 18d may be simply referred to as “electrode 18” when it is unnecessary to distinguish among them.

The distal end of a conductive wire (not illustrated) is connected to each electrode 18. The conductive wire is passed through the lumen of the shaft 10, and the proximal end is connected to a connector (not illustrated) of the handle 14 illustrated in FIG. 1. The power supply device 8 is electrically connected to each conductive wire through the connector of the handle 14. As elaborated later, a voltage is applied to the plurality of electrodes 18 by the power supply device 8.

The handle 14 is provided at the proximal end of the shaft 10, and is disposed outside the body during the use of the catheter 4 so as to be grabbed or operated by the operator. The handle 14 includes a main body part configured to be grabbed by the operator, and an operation unit configured to move the inner tube 22 back and forth. When the operation unit is operated, the inner tube 22 can be displaced to the proximal end side with respect to the shaft 10. In this manner, the electrode assembly 12 in a folded state is unfolded in the direction intersecting the axis of the shaft 10. In addition, when the operation unit is operated, the inner tube 22 can be displaced to the distal end side with respect to the shaft 10. As a result, the electrode assembly 12 in an unfolded state is folded. The connector is provided at the main body part. Note that the catheter 4 may include an irrigation mechanism for jetting irrigating fluid such as physiological saline from the distal end during ablation.

The counter electrode plate 6 is mounted on the body surface of the patient during ablation. In addition, the counter electrode plate 6 is electrically connected to the power supply device 8. During ablation, a voltage is applied to each electrode 18 and the counter electrode plate 6, thereby performing ablation.

The power supply device 8 includes an input unit 24, a power source unit 26, a control unit 28, and a display unit 30. The input unit 24 is composed of a dial, a button, a touch panel, and the like, and is operated by the operator of the ablation system 1. The operator can input to the power supply device 8 signals for instructing various setting values and operations via the input unit 24. Note that various setting values may be preliminarily set at shipment of products and the like and retained in the power supply device 8. The signals indicating setting values and the like are sent from the input unit 24 to the control unit 28.

The power source unit 26 applies an ablating voltage Vₒᵤₜ to the electrode 18 and the counter electrode plate 6 in accordance with a control signal CTL sent from the control unit 28. The power source unit 26 is composed of a predetermined power supply circuit such as a switching regulator, for example. The control unit 28 controls the entire operation of the power supply device 8, and executes a predetermined arithmetic process. The control unit 28 is composed of a microcomputer and the like, for example. The control unit 28 controls the application of the voltage Vout to the electrode 18 and the counter electrode plate 6 by sending the control signal CTL to the power source unit 26. The display unit 30 externally displays various information. The display unit 30 is composed of a liquid crystal display, a CRT display, an organic EL display, and the like.

Next, details of the control executed by the control unit 28 are described. The ablation system 1 of the present embodiment performs ablation on the affected area 2 by irreversible electroporation (IRE). Since IRE is non-thermal, it can suppress damage to tissues and nerves located around the affected area 2. For example, when performing pulmonary vein isolation to treat atrial fibrillation, it is possible to suppress damage to the esophagus and phrenic nerve around the affected area, thereby preventing complications such as esophageal fistula and phrenic nerve paralysis.

In IRE, pulsed electric field ablation (PFA) is performed. PFA is an ablation technique that destroys cells by means of a pulsed electric field generated by applying a high voltage between each electrode 18 and the counter electrode plate 6, thereby forming a lesion in the affected area 2. An electric field tends to reflect at the boundary between tissues. Therefore, when ablating the affected area, it is possible to suppress damage to adjacent tissues.

In the state where the electrode assembly 12 is inserted into the body of the patient through a blood vessel or the like and disposed at the affected area 2, the control unit 28 controls the power source unit 26 to sequentially apply voltage to each electrode 18 in accordance with the rules described below. Specifically, the control unit 28 controls the power source unit 26 to continuously apply voltage a plurality of times to some electrodes 18, and then continuously apply voltage a plurality of times to other electrodes 18. “Some electrodes 18” and “other electrodes 18” may each refer to either a single electrode 18 or to two or more electrodes 18. In addition, “continuously apply voltage a plurality of times” means applying biphasic pulses a plurality of times to the same electrode 18 without intervening voltage application to other electrodes 18. Hereinafter, a plurality of continuous voltage applications to the same electrode 18 will be simply referred to as “continuous application.” The fact that voltage is sequentially and continuously applied to each electrode 18 can be confirmed, for example, by connecting each electrode 18 to an oscilloscope.

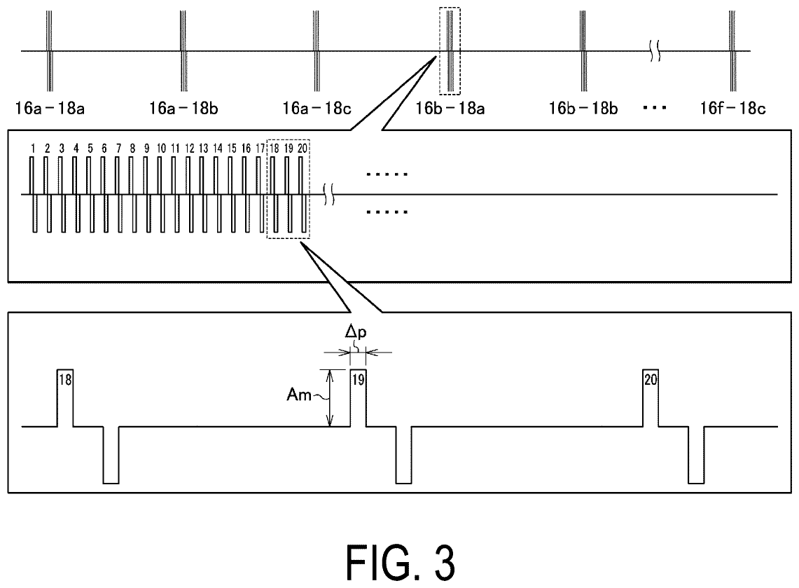

FIG. 3 is a diagram illustrating a first example of a timing of voltage application to each electrode 18. As an example, the control unit 28 sequentially and continuously applies voltage to the first electrode 18a to the third electrode 18c provided in one spline 16. Subsequently, the control unit 28 sequentially and continuously applies voltage to the first electrode 18a to the third electrode 18c provided in other splines 16. The control unit 28 repeats this sequential application, and finally applies voltage to all the first electrode 18a to the third electrode 18c provided in the electrode assembly 12. In the present embodiment, the fourth electrode 18d on each spline 16 is excluded from the voltage application targets. The fourth electrode 18d is provided for use in potential measurement or as a reserve in cases where the ablation range is wide. The fourth electrode 18d may be omitted.

More specifically, first, voltage is continuously applied a plurality of times to the first electrode 18a on the first spline 16a. Next, voltage is continuously applied a plurality of times to the second electrode 18b on the first spline 16a. Next, voltage is continuously applied a plurality of times to the third electrode 18c on the first spline 16a. Subsequently, the voltage application target is shifted to the second spline 16b, and voltage is continuously applied a plurality of times sequentially to the first electrode 18a to the third electrode 18c on the second spline 16b. Thereafter, voltage is also continuously applied a plurality of times sequentially to each electrode 18 on the third spline 16c to the sixth spline 16f.

The power source unit 26 of the present embodiment applies voltage to each electrode 18 so as to generate biphasic pulses (bipolar pulse). Accordingly, a positive voltage phase pulse and a negative voltage phase pulse are applied to each electrode 18, and the polarity of each electrode 18 alternates. An amplitude value Am of the voltage is 1000 V to 4000 V, for example. A pulse width Δp is 0.1 μs to 100 μs, for example.

In the present embodiment, voltage is sequentially and continuously applied to all the electrodes 18 that are the voltage application targets in the electrode assembly 12, namely the first electrode 18a to the third electrode 18c in the first spline 16a to the sixth spline 16f, and then the ablation is completed. Note that voltage application to all the electrodes 18 that are the voltage application targets may be defined as one cycle, and ablation may be completed by applying voltage over a plurality of cycles. The number of continuous voltage applications and the number of cycles may be appropriately set based on experiments or the like by the designer. For example, the number of continuous applications may be set within a range of 2 to 100. In addition, the number of continuous applications may differ for each electrode. The number of cycles is, for example, 1 to 1000. In addition, the control unit 28 may control the power source unit 26 to repeat sequential voltage application over a plurality of sets, with each set composed of a plurality of cycles of voltage application. The number of sets may be appropriately set based on experiments or the like by the designer, and is, for example, 1 to 100.

By sequentially applying voltage to each electrode 18, it is possible to increase the size of the lesion that can be formed by each electrode 18 in comparison with the case where voltage is simultaneously applied to all the plurality of electrodes 18. This is considered to be because, when voltage is collectively applied to all the plurality of electrodes 18, the current is distributed among the electrodes 18, resulting in a lower current density, whereas when voltage is applied sequentially to each electrode 18, the current is concentrated on a single electrode 18, increasing the current density. In addition, by sequentially applying voltage to a plurality of electrodes 18 with intervals, it is possible to suppress patient body movement caused by PFA.

In addition, by continuously applying voltage to the same electrode 18, the number of operations of switching the feeding target can be reduced in comparison with the case where the feeding target is switched for each single voltage application. In this manner, the control executed by the control unit 28 can be simplified.

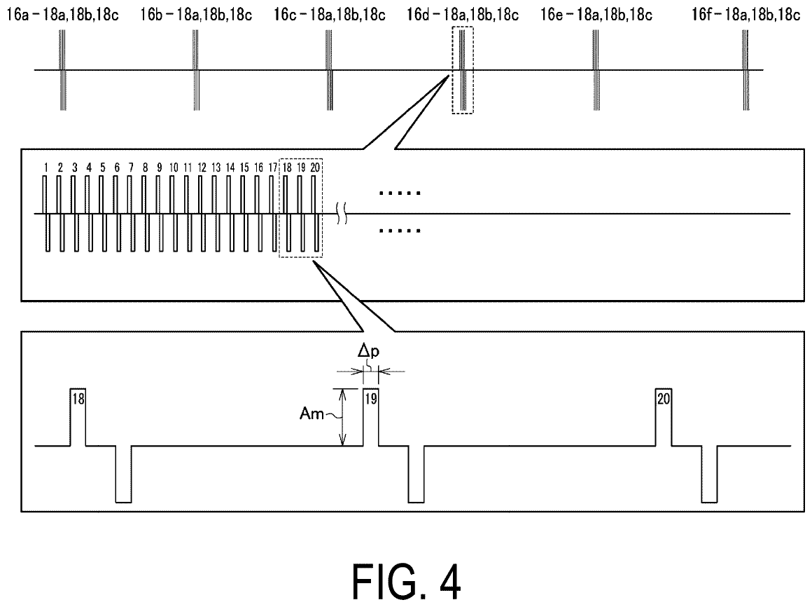

In addition, the control unit 28 may control the power source unit 26 in the following manner. FIG. 4 is a diagram illustrating a second example of a timing of voltage application to each electrode 18. Specifically, the control unit 28 controls the power source unit 26 to continuously apply voltage a plurality of times to all the electrodes 18 that are the voltage application targets provided in some splines 16, and then continuously apply voltage a plurality of times to all the electrodes 18 that are the voltage application targets provided in other splines 16. “Some splines 16” and “other splines 16” may each refer to either a single spline 16 or to two or more splines 16.

As an example, the control unit 28 simultaneously and continuously applies voltage to the first electrode 18a to the third electrode 18c provided in one spline 16. Subsequently, the control unit 28 simultaneously and continuously applies voltage to the first electrode 18a to the third electrode 18c provided in another spline 16. The control unit 28 repeats this sequential application, and finally applies voltage to all the first electrode 18a to the third electrode 18c provided in the electrode assembly 12. In the present disclosure, “simultaneous application” means that the states in which voltage is applied to each electrode 18 overlap at least partially in time.

More specifically, first, voltage is continuously applied a plurality of times simultaneously to the first electrode 18a to the third electrode 18c on the first spline 16a. Next, the voltage application target is shifted to the second spline 16b, and voltage is continuously applied a plurality of times simultaneously to the first electrode 18a to the third electrode 18c on the second spline 16b. Thereafter, the voltage application target is shifted in the order of the third spline 16c to the sixth spline 16f, and voltage is continuously applied a plurality of times simultaneously to the first spline 16a to the third electrode 18c of each spline 16. Note that voltage application to all the splines 16 may be defined as one cycle, and ablation may be completed by applying voltage over a plurality of cycles. In addition, it is possible to perform voltage application over a plurality of sets, with each set composed of a plurality of cycles of voltage application. The number of continuous voltage applications, the number of cycles, and the number of sets are as described above.

In this manner, even by switching the voltage application target on a spline-by-spline basis, it is possible to increase the size of the lesion that can be formed by each electrode 18, similarly to the first example in which the voltage application target is switched on an electrode-by-electrode basis. In addition, in comparison with the first example, the control can be further simplified. In addition, the power supply circuit for switching the voltage application target can be easily simplified. In this manner, the size of the power supply device 8 can be reduced. Further, the addition of electrodes 18 becomes easier. In addition, in comparison with the first example, the time required for ablation can be shortened. Alternatively, if the time until ablation is completed is kept the same, the number of voltage applications can be increased, thereby allowing for the formation of a larger lesion.

Note that the order of feeding is not limited as long as voltage is finally applied to all the electrodes 18 that are the feeding targets in the electrode assembly 12. For example, after voltage has been continuously applied to some electrodes 18 that are the feeding targets on one spline 16, the feeding target may be shifted to the electrode 18 on the next spline 16. As an example, after voltage is sequentially and continuously applied to the first electrode 18a on each spline 16, voltage may be sequentially and continuously applied to the second spline 16b on each spline 16, and finally voltage may be sequentially and continuously applied to the third spline 16c on each spline 16.

In addition, the order of the feeding to the first electrode 18a to the third electrode 18c on each spline 16 is not limited. The order of power feeding to the first electrode 18a to the third electrode 18c may differ for each spline 16. In addition, the order of the feeding to the first spline 16a to the sixth spline 16f is also not limited. Further, some splines 16 may be excluded from the feeding target. In addition, the plurality of electrodes 18 may be divided into a plurality of electrode groups, the number of which is fewer than the number of electrodes 18, and voltage may be sequentially and continuously applied to each electrode group. The above-described second example corresponds to an example of this grouping. The plurality of electrodes 18 belonging to each electrode group may be disposed on different splines 16. In addition, the electrodes 18 on a single spline 16 may belong to different electrode groups. In addition, the same electrode 18 may be assigned to two or more different electrode groups.

In addition, the shapes and numbers of the spline 16 and the electrode 18 are also not limited. In addition, the catheter 4 may not have the spline 16 at the distal end of the shaft 10, and the electrode 18 may be disposed on the shaft 10. In addition, the power source unit 26 may apply voltage to each electrode 18 so as to generate monophasic pulses.

In addition, the configurations of the catheter 4 and the power supply device 8 may be appropriately changed. For example, in the catheter 4, the distal end side of the shaft 10 may be curved in one or more directions by operating the handle 14. The control of the power source unit 26 by the control unit 28 may be implemented by hardware (circuits) or by software (programs). When implemented by software, the software is composed of a group of programs for causing a computer to execute the respective functions. Each program may, for example, be preinstalled in the computer, or installed in the computer via a network or from a recording medium.

The embodiments of the present disclosure have been described in detail. The above-described embodiments are merely specific examples for implementing the present disclosure. The contents of the embodiments do not limit the technical scope of the present disclosure, and various design modifications such as changes, additions, or deletions of components are possible within the scope of the inventive concept defined in the claims without departing from the spirit of the present disclosure. A newly modified embodiment with design changes incorporates the respective effects of the combined embodiments and modifications. In the above-described embodiments, expressions such as “of the present embodiment” or “in the present embodiment” are used to emphasize the contents where such design modifications are possible, but design modifications are also permissible for contents without such expressions. Any arbitrary combination of components included in the respective embodiments is also a valid aspect of the present disclosure. The hatching indicated in the cross-sections of the drawings does not limit the material of the hatched object.

The embodiments may also be specified by the items described below.

### Item 1

A power supply device (8) including:
a power source unit (26) electrically connected to a catheter (4) including a plurality of electrodes (18), and configured to apply voltage to the plurality of electrodes (18); and
a control unit (28) configured to control the power source unit (26) to continuously apply voltage a plurality of times to one or more electrodes (18) of the plurality of electrodes (18), and then continuously apply voltage a plurality of times to another one or more electrodes (18) of the plurality of electrodes (18).

### Item 2

The power supply device (8) according to item 1, in which
the catheter (4) includes a shaft (10) and a plurality of splines (16) disposed in an axial direction of the shaft (10),
each spline is provided with at least one electrode (18) of the plurality of electrodes (18), and
the control unit (28) controls the power source unit (26) to continuously apply voltage a plurality of times to all the electrodes (18) that are voltage feeding targets provided in one or more splines of the plurality of splines (16), and then continuously apply voltage a plurality of times to all the electrodes (18) that are voltage feeding targets provided in another one or more splines of the plurality of splines (16).

### Item 3

An ablation system (1) including:
a catheter (4) including a plurality of electrodes (18); and
the power supply device (8) according to item 1 or 2.

What is claimed is:

1. A power supply device comprising:
a power source unit electrically connected to a catheter including a plurality of electrodes, and configured to apply voltage to the plurality of electrodes; and
a control unit configured to control the power source unit to continuously apply voltage a plurality of times to one or more electrodes of the plurality of electrodes, and then continuously apply voltage a plurality of times to another one or more electrodes of the plurality of electrodes, wherein
the catheter includes a shaft and a plurality of splines disposed in an axial direction of the shaft,
each spline is provided with at least one electrode of the plurality of electrodes, and
the control unit controls the power source unit to continuously apply voltage a plurality of times to all the plurality of electrodes that are voltage application targets provided in one or more splines of the plurality of splines, and then continuously apply voltage a plurality of times to all the plurality of electrodes that are voltage application targets provided in another one or more splines of the plurality of splines.

2. (canceled)

3. An ablation system comprising:
a catheter including a plurality of electrodes; and
the power supply device according to claim 1.
